(19) United States
(12) Patent Application Publication
(10) Pub. No.: US 2005/0085728 A1
(43) Pub. Date: Apr. 21, 2005

(54) Apparatus and program for estimating viscoelasticity of soft tissue using ultrasound

(75) Inventor: Osamu Fukuda, Ibaraki (JP)

Correspondence Address: BAKER & BOTTS, 30 ROCKEFELLER PLAZA, NEW YORK, NY, 10112, US

(73) Assignee: National Institute of Advanced Industrial Science and Technology, Tokyo (JP)

(21) Appl. No.: 10/968,604

(22) Filed: Oct. 19, 2004

(30) Foreign Application Priority Data
Oct. 20, 2003 (JP) 2003-359385
Oct. 13, 2004 (JP) 2004-298519

(51) Int. Cl. A61B 8/00

(52) U.S. Cl. 600/449

(57) ABSTRACT

The present invention allows even soft tissue such as body tissue having a hierarchic structure of skin, fat, muscle and bone, etc., to be estimated and allows estimation only through a short-time pressing operation to thereby reduce damages to the soft tissue. The present invention is constructed of an ultrasonic probe for transmitting/receiving an ultrasonic signal, a target deformation amount calculation section for calculating an amount of deformation of a target shape from a time variation of data received from the ultrasonic probe, a movement mechanism for moving the ultrasonic probe, a probe control section for controlling the probe, a position sensor for measuring the position of the probe, a force sensor for measuring a force applied to the probe section, a viscoelasticity estimation section for estimating viscoelasticity of the target based on values obtained from the position sensor, force sensor, target deformation amount calculation section and a viscoelasticity display section for presenting the estimated viscoelasticity to the user.

### FIELD OF THE INVENTION

The present invention relates to an apparatus and method for estimating impedance values of tissue (elasticity, viscosity, inertia) based on information on an ultrasonic signal transmitted/received from the surface of soft tissue.

### BACKGROUND INFORMATION

There is a proposal on a method of pressing a force sensor against the surface of soft tissue and estimating viscoelasticity of the tissue from a relationship between the pressing distance and the force measured by the force sensor. (see e.g., JP Patent Publication (Kokai) No. 08-29312A (1996).

However, this method performs estimation on assumption that the target tissue has uniform viscoelasticity in various regions and when, for example, a target, only the surface of which is covered with a hard film, is estimated, a large estimation error occurs.

Furthermore when the target tissue has a hierarchic structure such as skin, fat, muscle, bone as in the case of a human body, for example, it has been extremely difficult to estimate viscoelasticity of the respective regions of each tissue.

On the other hand, there is another method of pressing an ultrasonic probe against soft tissue such as body tissue so as to deform the soft tissue and measuring elasticity of the tissue from the force applied and the amount of deformation at this time. (see e.g., JP Patent Publication (Kokai) No. 05-317313B (1993), JP Patent Publication (Kohyo) No. 2001-519674A).

However, the proposed conventional methods only incorporate a physical model describing the relationship between a force and amount of deformation of the target tissue in a steady state and do not consider a transient variation (that is, a non-steady state variation) of a reaction force which the target tissue responds to the movement, etc., of the probe position. For this reason, it has been extremely difficult to estimate viscosity and inertia of soft tissue.

Furthermore, since after the pressing (movement) of the probe ends until the reaction force from the tissue reaches a steady state, forces are caused by viscosity and inertia, the conventional methods have been unable to perform accurate estimation and estimation requires a certain degree of time. For this reason, there have been some cases where irreversible shape variations are provoked in the target soft tissue while maintaining the state in which the probe is pressed.

### SUMMARY OF THE INVENTION

The present invention has been implemented taking into account the problems described above and it is an object of the present invention to provide a method, apparatus and program capable of estimating elasticity, viscosity and inertia of soft tissue such as body tissue having a hierarchic structure of skin, fat, muscle and bone, etc., layer by layer and reducing damages to the soft tissue by allowing estimation through only a short-time pressing operation.

In order to attain the above described object, the present invention provides a method, apparatus and program for estimating viscoelasticity of soft tissue including an ultrasonic probe for transmitting/receiving an ultrasonic signal, a target deformation amount calculation section for calculating an amount of deformation in the target shape from a time variation of data received at ultrasonic probe, a movement mechanism for moving the ultrasonic probe, a probe control section for controlling the movement mechanism, a position sensor for measuring the position of the probe, a force sensor for measuring a force applied to the probe section, a viscoelasticity estimation section for estimating viscoelasticity of the target based on values obtained from the position sensor, force sensor and target deformation amount calculation section, and a viscoelasticity display section for displaying the estimated viscoelasticity to the user.

The method, apparatus and program for estimating viscoelasticity of soft tissue using ultrasound according to the present invention measure an amount of deformation at various regions and layers of tissue using ultrasonic signal, combine the measurement result with separately measured information from the force sensor and position sensor, allows estimation of elasticity, viscosity and inertia according to a physical model which describes the relationship between a force and an amount of deformation of a target, allows estimation through only a short-time pressing operation to thereby reduce damages to soft tissue.

### BRIEF DESCRIPTION OF THE DRAWINGS

FIG. 1 is a block diagram schematically showing a system of the present invention;

FIG. 2 is a schematic diagram of movement of an ultrasonic probe and deformation of soft tissue in viscoelasticity estimation of the present invention;

FIG. 3 is an example of variations of a force sensor; position sensor in the system of the present invention;

FIG. 4 is an example of a physical model used in viscoelasticity estimation of the present invention; and

FIG. 5 is an example of information measured by the system of the present invention.

### DETAILED DESCRIPTION OF THE PREFERRED EMBODIMENTS

The present invention is intended to provide a method, apparatus and program capable of estimating elasticity, viscosity and inertia of soft tissue such as body tissue having a hierarchic structure of skin, fat, muscle and bone, layer by layer and reducing damages to the soft tissue by allowing estimation through only a short-time pressing operation and measures three pieces of information; a shape variation of target tissue obtained from an ultrasonic signal, an amount of movement of the probe and a force applied, with high accuracy and a simple structure.

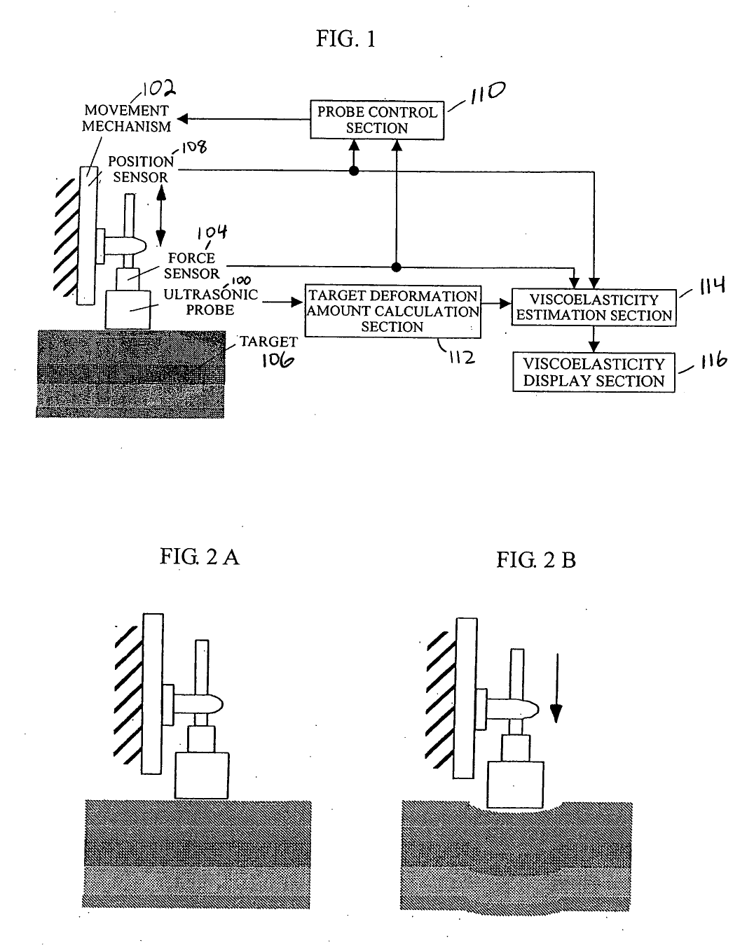

With reference now to the attached drawings, an exemplary embodiment of the present invention will be explained in detail below. FIG. 1 is a block diagram schematically showing a system of the present invention. As illustrated in this figure, an ultrasonic probe 100 is fixed to a movement mechanism 102 (linear slider) fixed to an absolute system through a force sensor 104 (load cell) and can measure a reaction force from a target tissue 106 produced by movement. Furthermore, an amount of movement of the ultrasonic probe 100 can be measured using a position sensor 108 (encoder) incorporated in the movement mechanism 102 (linear slider) at high sampling intervals (1 msec).

FIG. 2 is a schematic diagram of movement of an ultrasonic probe and deformation of soft tissue in viscoelasticity estimation of the present invention. As illustrated in the figure, a target 106 is deformed when the probe is pressed into the target 106. FIG. 2A shows the target before the probe is pressed and FIG. 2B shows the target when the probe is pressed. If the target has a hierarchic structure, it is possible to observe deformation of each layer.

The probe control section 110, target deformation amount calculation section 112, viscoelasticity estimation section 114 and viscoelasticity display section 116 are constructed using a personal computer and software program. Use of a personal computer facilitates data management, etc., and provides a high degree of convenience, but when miniaturization is preferred, it is also possible to construct a built-in type apparatus using a one-board type computer, PLD, FPGA, PIC, etc., depending on the purpose of use. Details of the respective sections will be explained below.]

The following paragraphs have particular relevance to the ultrasonic probe.

As the ultrasonic probe 100, one incorporating one channel of piezoelectric element is used. This probe 100 can measure one-dimensional data and when the probe 100 is contacted with a target 106, one-dimensional data is obtained in the depth direction on the same line as the direction in which the probe 100 moves. The oscillation frequency of the ultrasound element is 3.5 MHz and is appropriate to measure viscoelasticity (acoustic impedance) of a human body, etc. However, the number of channels and the oscillation frequency of the element may also be selected arbitrarily according to the target tissue. For example, when the number of channels is increased, it is possible to obtain two-dimensional data (image), three-dimensional data (volume) and analyze this data.

In this way, the ultrasonic probe 100 has the function capable of selecting the frequency band, focus position, etc., of an ultrasonic signal according to the target tissue and select or use them simultaneously as required.

The following paragraphs have particular relevance to the target deformation amount calculation section 112.

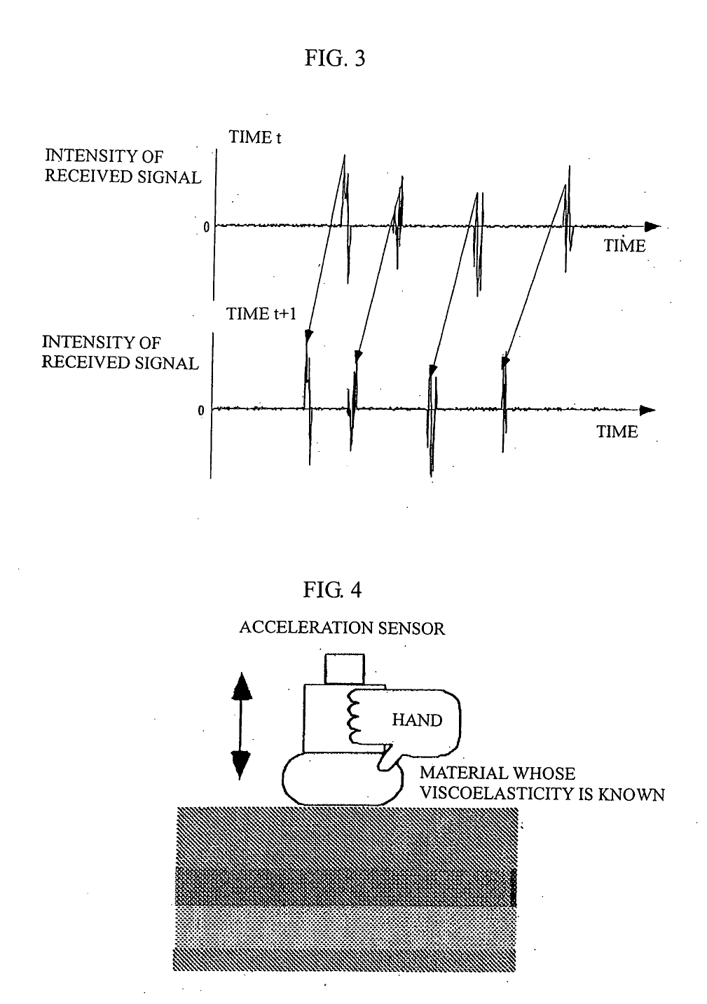

FIG. 3 is a schematic diagram of a signal received by an ultrasonic probe. The ultrasonic signal is strongly reflected at a point at which viscoelasticity (acoustic impedance) changes in the target tissue, and therefore a variation of amplitude reflecting the change appears in the signal. Here, when a case where the ultrasonic probe is pressed into the target and the shape of the target is deformed is considered, a shift is observed in the shape of the signal received at time t and time t+1.

In the example of time in FIG. 3, a time difference between time t and time t+1 is 1 [msec] and a maximum value on the horizontal axis of each graph is approximately 50 [μsec]. However, this value can be freely adjusted by the moving speed of the probe and when, for example, the probe is moved slowly, it is not necessary to shorten the time interval so much, whereas when the probe is moved fast, a shorter time interval is preferable.

Thus, it is possible to divide the pattern of the received signal into small segments on the time axis and calculate to which part in the pressing direction (one-dimensional) a certain segment at time t has moved at time t+1 by calculating a correlation value on a pattern at time t+1 and find how the target has deformed. At this time, deformation calculated from soft parts is large while deformation calculated from hard parts is small. Further carrying forward the processing to times t+1, t+2, t+3, . . . , sequentially makes it possible to obtain changes of various regions caused by the pressing of the ultrasonic probe as a time series.

In the case where the ultrasonic probe has a plurality of channels and two-dimensional image data and three-dimensional image data can also be received, completely the same processing can be executed. That is, target data at time t and t+1 is prepared, data in the target space is divided into small areas and it is measured to which part the respective segments move (two-dimensional, three-dimensional).

The following paragraphs have particular relevance to the movement mechanism, probe control section, and position sensor.

The movement mechanism 102 is used for causing the ultrasonic probe 100 to carry out a pressing operation such that the target 106 is deformed. The probe control section 110 is used for setting a target position or target force that varies with time in response to the physical characteristics or deformation of the target 106, and for feedback-controlling the movement mechanism using reception data from the position sensor, force sensor, and the ultrasonic probe individually or using information combining such individual data, such that the target position or target force is followed. The position sensor 108 is used for measuring the position of the probe.

A linear motor table is used for the movement mechanism 102. When the probe position is controlled, there are methods such as performing position control and performing force control. When position control is performed, the track is controlled so that the variation in acceleration becomes a minimum. Since inertia of the probe 100 itself caused by the movement is also superimposed on the force sensor 104 for measuring a force applied to the probe 100, this track control is performed to suppress the influence of the inertia. When force control is performed, the force is controlled so that the force generated between the target and ultrasonic probe 100 becomes a target value. When the target 106 is fragile, position control may damage the target 106 due to an excessive force generated, but in the case of force control, no force exceeding the set target value is generated, and therefore it is possible to avoid the danger.

For the movement mechanism, not only the linear motor table but also a variety of mechanisms such as a motor and ball screw, electromagnetic drive mechanism, mechanical spring mechanism, air-pressure, shape-memory alloy (bimetal) can be used. For example, to realize weight reduction, size reduction or cost reduction, it is possible to exclude an actuator of a motor, etc., by adopting a mechanical spring mechanism. In this case, there is no need to supply power, etc., to the movement mechanism 102.

The probe control section 110 can generate a signal to drive the movement mechanism, give a command to the drive mechanism and realize feedback control on a target movement position or target power generated with reference to values at the position measuring section and force measuring section as required. Furthermore, when the ultrasonic probe 100 is moved the movement mechanism 102 and probe control section 110 can adopt movement tracks so that an acceleration variation perk) thereof becomes a minimum for the purpose of improving estimation accuracy.

Furthermore, it is also possible to manually carry out the pressing operation without using any movement mechanism (see FIG. 4). In this case, it is possible to measure the position using an acceleration sensor or spatial position sensor for the ultrasonic probe or using a fixed laser rangefinder fixed to an absolute system and information from a CCD camera. Furthermore, since reception data from the ultrasonic probe also includes distance-related information, there is also a method of calculating an amount of movement of the probe from the information.

For the position sensor, it is possible to use an encoder provided for the movement mechanism, an acceleration sensor fixed to the ultrasonic probe or a spatial position sensor fixed to the ultrasonic probe or further a laser rangefinder fixed to an absolute system, CCD camera, etc.

Generally, when determining the viscoelasticity of tissue, vibrations are often caused in the tissue if a deforming load is applied to the tissue in a sudden manner, possibly leading to a decrease in estimation accuracy or the destruction of the tissue. In particular, care should be taken in the case of a tissue with a high specific gravity and a large elasticity. On the other hand, in order to produce a large reaction force in tissues with small elasticity or viscosity, it is necessary to produce a large deformation, cause a deformation to be produced at a fast rate, or devise some other measures. In such cases, it is also necessary to apply or remove load in such a manner as to vary acceleration or deceleration in a gradual manner, in order to avoid the decrease in estimation accuracy or the destruction of the tissue.

In many tissues, elasticity or viscoelasticity varies nonlinearly in response to deformation, and such characteristic changes often differ depending on the stage of deformation. For this reason, in cases where the target is an object in which a plurality of tissues coexist, it is desirable to control the pressing position, pressing rate, and pressing force as needed in an effective manner depending on each stage of deformation. Namely, it is not sufficient just to control the pressing amount, pressing rate, and pressing force during the probe-pressing operation, and rather it is necessary to feedback-control their temporal changes, namely the target position or target force at each time, in the interval between the start and finish of operation of the movement mechanism, so that they can be controlled and utilized with speed and accuracy.

An example of probe movement control in which estimation is difficult is a vibratory operation. When a target consisting of a plurality of tissues with different viscoelasticity characteristics is vibrated, each tissue normally deforms in a complex manner in each period due to the interaction of transient deformation of each. In such a case, it is often difficult to estimate the elasticity, viscosity, and inertia of each tissue accurately based on the information about the instantaneous deformation and force.

An example of probe movement in which estimation is easy is a “pressing” operation. In order to eliminate the influence of the complicated vibrations due to the interaction of multiple viscoelastic tissues, it is desirable to control the movement of the probe during the interval between the state in which transient deformation of the target is occurring and the steady state.

When the probe or force sensor is moved, inertia is produced by their own acceleration, and if the rate of change in acceleration or deceleration is high, their inertia could be expected to have a large influence on measurement values (position sensor, force sensor), resulting in a decrease in estimation accuracy for elasticity, viscosity, and inertia. For example, when estimating the inertia of a tissue with a small specific gravity, a large acceleration is required in order to produce a large inertia. In such a case too, it is also desirable to accelerate or decelerate smoothly rather than sharply.

In order to obtain a smooth change of acceleration, the movement should be controlled in such a manner as to minimize the value obtained by integrating the jerk, which is a time-differentiated value of acceleration, with respect to control time. Namely, in the following mathematical expressions: and

$$\begin{matrix}C = \frac{1}{2} {\int }_{0}^{{t}_{f}} \frac{{ⅆ}^{3} x}{ⅆ {t}^{3}} ⅆ t & ( 1 ) \\ and & \\ x ( t ) = x ( 0 ) + \{ x ( 0 ) - x ( {t}_{f} ) \} ( 15 {t}_{s}^{4} - 6 {t}_{s}^{5} - 10 {t}_{s}^{3} ) & ( 2 )\end{matrix}$$

where C is evaluation value, _{if} is end time of movement control, tₛ is unit control time (0, . . . , 1) and x is the position of the probe, the trajectory of movement x should be controlled such that evaluation value C in expression (1) becomes minimum. In this case, the trajectory of movement of the probe is expressed by expression (2).

Thus, in the probe control section, in order to obtain best results in the estimation of elasticity, viscosity and inertia, distance-related information calculated from the reception data from the position sensor, force sensor, or the probe is utilized so that the probe can be feedback-controlled at high speed and accurately. Specifically, a target position and target force are set that vary with time in response to the physical characteristics or deformation of the target, and a feedback control is effected in accordance with the thus set position and force. It goes without saying that, when the target is limited in advance, the mechanism may be designed such that a predetermined distance, speed or acceleration is realized at each time. In this way, it becomes possible to estimate the characteristics of each tissue accurately in an object in which a plurality of tissues with different viscoelasticity coexist.

The following paragraphs have particular relevance to the force sensor.

For the force sensor 104, a load cell is used. This sensor allows a reaction force to be measured when the ultrasonic probe 100 is pressed into a target 106. The sensor 104 can be a distortion gauge type sensor or piezoelectric type force sensor, but when elasticity of the target 106 is large, etc., one capable of measuring up to a high frequency band is preferable. This is intended to measure high-frequency vibration with accuracy.

Furthermore, it is also possible to insert an object whose viscoelasticity is known between the ultrasonic probe and target as shown in FIG. 4 and calculate backward a force from the amount of deformation thereof. Or it is also possible to insert a small bag containing a liquid instead of the object and measure the pressure applied to the liquid therein.

The following paragraphs have particular relevance to the visoelasticity estimation section.

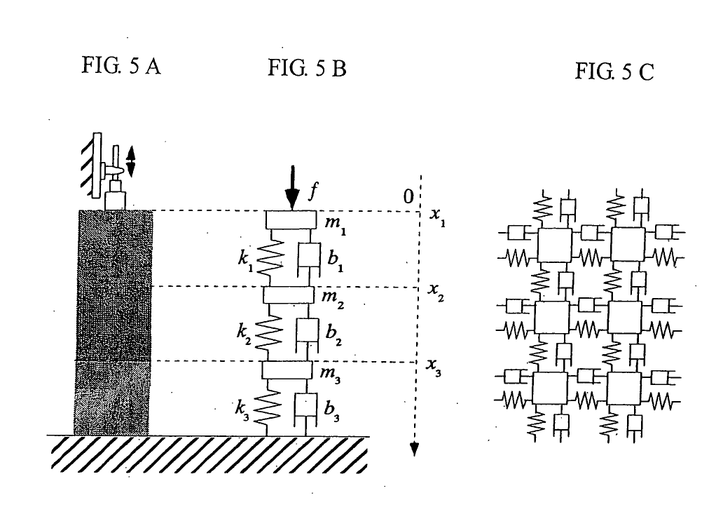

In the viscoelasticity estimation section 114, the elasticity, viscosity, and inertia at each part of the target tissue are estimated on the basis of the measurement values obtained from the position sensor, force sensor, and the target deformation amount calculation section individually that include transient changes. FIG. 5 shows an example of a physical model used in viscoelasticity estimation of the present invention. This figure expresses a target having a hierarchic structure as shown in 5A using a physical model consisting of elasticity, viscosity and inertia in 5B. Similar modeling is also possible for a multi-dimensional model and, for example, in the case of a two-dimensional model, modeling shown in 5C is available.

Here, x₁, x₂, x₃ indicate boundary positions in the target layer measured by the ultrasonic probe and amounts of movement of these positions can be calculated by the target deformation amount calculation section. m₁, m₂, m₃ are masses in the respective areas, k₁, k₂, k₃ are elastic coefficients in the respective areas, b₁, b₂, b₃ are viscosity coefficients in the respective areas and f denotes a force of the probe applied to the target.

At this time, an equation of motion of a physical model describing a relationship between the elasticity, viscosity, inertia of the target, force applied to the target and the amount of deformation of the target is given as follows:
m₁\ddot{x}₁+b(\dot{x}₁−\dot{x}₂)+k₁(x₁−x₂)=f
m₂\ddot{x}₂+b₂(\dot{x}₂−\dot{x}₃)+k₂(x₂−x₃)−b₁(\dot{x}₁−\dot{x}₂)−k₁(x₁−x₂)=0
m₃\ddot{x}₃+b₃\dot{x}₃+k₃x₃−b₂(\dot{x}₂−\dot{x}₃)−k₂(x₂−x₃)=0

However, this formula applies to a case where elasticity and viscosity of the target are constant and act on the amount of deformation linearly. These parameters may vary depending on the target and may often act on the amount of deformation non-linearly, and in this case, the parameters can be expressed as follows:
m₁\ddot{x}₁+b₁(\dot{x}₁−\dot{x}₂)^{q1}+k₁(x₁−x₂)^{p1}=f
m₂\ddot{x}₂+b₂(\dot{x}₂−\dot{x}₃)^{q2}+k₂(x₂−x₃)^{p2}−b₁(\dot{x}₁−\dot{x}₂)^{q1}−k₁(x₁−x₂)^{p1}=0
m₃\ddot{x}₃+b₃\dot{x}₃^{q3}+k₃x₃^{p3}−b₂(\dot{x}₂−\dot{x}₃)^{q2}−k₂(x₂−x₃)^{p2}=0
where p₁, p₂, p₃ are elasticity exponents and q₁, q₂, q₃ are viscosity exponents.

In the above described formulas, since x₁, x₂, x₃ and first-degree differential, second-degree differential thereof and f are known values measured from the target deformation amount calculation section, position sensor and force sensor, there are nine unknown quantities of m1, m2, m3, b1, b2, b3, k1, k2, k3 in Formula 1, while there are 15 unknown quantities of m1, m2, m3, b1, b2, b3, k1, k2, k3, q1, q2, q3, p1, p2, p3 in Formula 2. However, known parameters are time variable and can be measured at respective times, and therefore it is possible to form simultaneous equations corresponding to the measuring time. Therefore, it is also possible to calculate unknown parameters in a manner similar to a numerical analysis, and as described above it is possible to estimate elasticity, viscosity and inertia of the target.

Thus, the viscoelasticity estimation section 114 incorporates equations of motion of a physical model describing a relationship between the elasticity, viscosity, inertia of the target, force applied to the target and amount of deformation of the target and it is possible to estimate values of elasticity, viscosity and inertia in various regions of the target tissue from the values measured at the position sensor, force sensor and target deformation amount calculation section.

The following paragraphs have particular relevance to the viscoelasticity display section.

The values estimated by the viscoelasticity estimation section 114 are converted to a format easily understandable and presented to the user. In this case, according to the specification of the ultrasonic probe, for example, one-dimensional data is displayed as strip-shaped one-dimensional data, two-dimensional data is displayed as an image and three-dimensional data is displayed as a three-dimensional object. Here, applying the estimated values to a color tone and gradation provides a display in a format intuitively easy to understand. For example, applying elasticity, viscosity and inertia to color tones of RGB and applying magnitudes of the respective values to gradation makes visible the information included in these parameters. Elasticity, viscosity and inertia may also be displayed separately.

Furthermore, when areas in which values of elasticity, viscosity and inertia should be measured are known beforehand, the values related to the respective areas may also be displayed in numerical values.

The method, apparatus and program for estimating viscoelasticity of soft tissue using ultrasound according to the present invention can estimate elasticity, viscosity and inertia in respective regions and layers of the tissue and reduce damages to the soft tissue through only a short-time pressing operation, and therefore the following industrial applications can be considered.

Since the present invention can estimate viscoelasticity of tissue, hierarchic tissue inside soft tissue, the present invention is suitable for measurement of a human body (skin, fat, muscle, dropsy, blood vessel, organ, etc.). The present invention is also applicable to industrial fields such as medical equipment, beauty treatment, rehabilitation, health, sports, etc.

The present invention can perform viscoelasticity estimation on meat such as beef before shipment in a noninvasive manner and it is possible to apply the present invention to inspection of lipid and quality of meat based on the estimated values.

By carrying out viscoelasticity estimation when manufacturing soft materials such as silicon and rubber, etc., it is possible to inspect a mixture of foreign matters or bubbles inside.

Even when a target is too soft to be touched directly, it is possible to immerse the target in water and apply a water pressure thereto to deform it and estimate viscoelasticity thereof. It is possible to perform manufacturing inspection of a polymer material such as gel, etc.

While there have been described what are believed to be the preferred embodiments of the present invention, those skilled in the art will recognize that other and further changes and modifications may be made thereto without departing from the spirit of the invention, and it is intended to claim all such changes and modifications as fall within the true scope of the invention.

1. An apparatus for estimating viscoelasticity of soft tissue comprising:
an ultrasonic probe for transmitting and receiving an ultrasonic signal;
a target deformation amount calculation section for calculating an amount of deformation of a target shape from a time variation of data received from the ultrasonic probe;
a movement mechanism for causing the ultrasonic probe to perform a pressing operation such that the target is deformed;
a position sensor for measuring the position of the ultrasonic probe;
a force sensor for measuring a reaction force when the ultrasonic probe is pressed into the target;
a probe control section for setting a target position and target force which vary with time according to a physical characteristic and deformation of the target and feedback-controlling the movement mechanism using information on received data from at least one of the position sensor, the force sensor and the ultrasonic probe so as to follow said target position and target force;
a viscoelasticity estimation section for estimating viscoelasticity of the target based on measured values including transient variations obtained from the position sensor, force sensor and target deformation amount calculation section.

2. The apparatus of claim 1, further comprising a viscoelasticity display section for presenting the estimated viscoelasticity to a user.

3. The apparatus of claim 1, wherein the ultrasonic probe is provided with a single channel or a plurality of channels of piezoelectric elements capable of transmitting and receiving ultrasonic signals, can measure one-dimensional data or multi-dimensional data, has the function capable of selecting a frequency band of the ultrasonic signal and focus position according to the target tissue and selects or uses simultaneously the frequency band and focus position.

4. The apparatus of claim 1, wherein the target deformation amount calculation section calculates an amount of deformation of the shape of the target tissue from a time variation of the data measured by the ultrasonic probe.

5. The apparatus of claim 1, wherein the movement mechanism is provided with at least one of a motor and ball screw, linear motor, electromagnetic drive mechanism, mechanical spring mechanism, air pressure, and shape-memory alloy so as to move the ultrasonic probe by a predetermined distance, at predetermined speed or acceleration.

6. The apparatus of claim 1, wherein the probe control section generates a signal for driving the movement mechanism, gives a command to the drive mechanism and feedback-controls the movement position of the target and target force generated with reference to values of the position measuring section and force measuring section at any time as required.

7. The apparatus of claim 1, wherein when moving the ultrasonic probe, the movement mechanism and probe control section take a movement track so that the variation in acceleration becomes a minimum for the purpose of improving the estimation accuracy.

8. The apparatus of claim 1, wherein the position sensor comprises at least one of an encoder provided for the movement mechanism, an acceleration sensor fixed to the ultrasonic probe, a spatial position sensor fixed to the ultrasonic probe, a laser rangefinder fixed to an absolute system, and a CCD camera.

9. The apparatus of claim 1, wherein the force sensor comprises at least one of a distortion gauge type sensor attached to the apparatus, a load cell, a piezoelectric type force sensor, and a pressure sensor capable of measuring a pressure applied to a liquid contained in a small bag inserted between the ultrasonic probe and the target.

10. The apparatus of claim 1, wherein the viscoelasticity estimation section incorporates equations of motion of a physical model describing a relationship between elasticity, viscosity and inertia of the target and the force applied to the target and an amount of deformation of the target and estimates values of elasticity, viscosity and inertia in the respective sections of the target tissue from the values measured by the position sensor, force sensor and target deformation amount calculation section.

11. The apparatus of claim 10, wherein the viscoelasticity display section, for presenting the estimated viscoelasticity to the user, presents the values of elasticity, viscosity and inertia which have been estimated by the viscoelasticity estimation section and converted to color tones and gradation using a method visually easy to identify to the user or presents values of elasticity, viscosity and inertia about regions of the target tissue specified by the user in numerical values.

12. A computer readable medium for estimating viscoelasticity of soft tissue with an ultrasonic probe for transmitting and receiving an ultrasonic signal pressed against a target of soft tissue to deform the target and estimate viscoelasticity of the target from a relationship between the force applied thereto and the amount of deformation of the target, the computer readable medium having a set of instructions operable to direct a processor to perform the steps of:
setting a target position and target force which vary with time according to a physical characteristic and deformation of the target and feedback-controlling the movement mechanism using information on received data from at least one of a position sensor, a force sensor, and an ultrasonic probe so as to follow said target position and target force, causing the ultrasonic probe to perform a pressing operation in which deformation of the target changes transiently, measuring the position of said ultrasonic probe and measuring a reaction force when said ultrasonic probe is pressed into the target, and
calculating an amount of deformation of the target shape from a time variation of the data received from said ultrasonic probe and estimating viscoelasticity of the target based on measured values including the position of the ultrasonic probe, reaction force against the ultrasonic probe, transient variations of the amount of deformation of the target shape.

13. The computer readable medium of claim 12, wherein said ultrasonic probe comprises a single channel or a plurality of channels of piezoelectric elements capable of transmitting and receiving ultrasonic signals, can measure one-dimensional data or multi-dimensional data, has the function capable of selecting a frequency band of the ultrasonic signal and focus position according to the target tissue and selects or uses simultaneously the frequency band and focus position as required.

14. The computer readable medium of claim 12, wherein the amount of deformation of said target shape is calculated by calculating an amount of deformation of the shape of the target tissue from a time variation of the data measured by the ultrasonic probe.

15. The computer readable medium of claim 12, wherein in order to control the movement of said ultrasonic probe, at least one of a motor and ball screw, a linear motor, an electromagnetic drive mechanism, a mechanical spring mechanism, an air pressure, and a shape-memory alloy is used so as to move the ultrasonic probe by a predetermined distance, at predetermined speed or acceleration.

16. The computer readable medium of claim 12, wherein the movement of said ultrasonic probe is controlled by generating a drive signal, giving a command and performing feedback-control with respect to the target movement position and target force generated.

17. The computer readable medium of claim 12, wherein when moving the ultrasonic probe, the movement of said ultrasonic probe is controlled by taking a movement track so that the variation in acceleration becomes a minimum for the purpose of improving the estimation accuracy.

18. The computer readable medium of claim 12, wherein the position of said ultrasonic probe is measured using at least one of an encoder provided for the movement mechanism, an acceleration sensor fixed to the ultrasonic probe, a spatial position sensor fixed to the ultrasonic probe, a laser rangefinder fixed to an absolute system, and a CCD camera.

19. The computer readable medium of claim 12, wherein the force applied to said ultrasonic probe is measured using at least one of a distortion gauge type sensor attached to the apparatus, a load cell, a piezoelectric type force sensor, and a pressure sensor capable of measuring a pressure applied to a liquid contained in a small bag inserted between the ultrasonic probe and the target.

20. The computer readable medium of claim 12, wherein viscoelasticity is estimated based on equations of motion of a physical model describing a relationship between elasticity, viscosity and inertia of the target and the force applied to the target and an amount of deformation of the target by estimating values of elasticity, viscosity and inertia in the respective regions of the target tissue from the position of the ultrasonic probe, force applied and amount of deformation of the target.

21. The computer readable medium of claim 20, wherein the estimated values of elasticity, viscosity and inertia are converted to color tones and gradation, presented to a user using a method visually easy to identify or values of elasticity, viscosity and inertia about regions of the target tissue specified by the user are presented in numerical values.

22. A method for estimating viscoelasticity of soft tissue with an ultrasonic probe for transmitting and receiving an ultrasonic signal pressed against a target of soft tissue to deform the target and estimate viscoelasticity of the target from a relationship between the force applied thereto and the amount of deformation of the target, the method comprising:
setting a target position and target force which vary with time according to a physical characteristic and deformation of the target and feedback-controlling the movement mechanism using information on received data from at least one of a position sensor, a force sensor, and an ultrasonic probe so as to follow said target position and target force, causing the ultrasonic probe to perform a pressing operation in which deformation of the target changes transiently, measuring the position of said ultrasonic probe and measuring a reaction force when said ultrasonic probe is pressed into the target, and
calculating an amount of deformation of the target shape from a time variation of the data received from said ultrasonic probe and estimating viscoelasticity of the target based on measured values including the position of the ultrasonic probe, reaction force against the ultrasonic probe, transient variations of the amount of deformation of the target shape.

23. The method of claim 22, wherein said ultrasonic probe comprises a single channel or a plurality of channels of piezoelectric elements capable of transmitting and receiving ultrasonic signals, can measure one-dimensional data or multi-dimensional data, has the function capable of selecting a frequency band of the ultrasonic signal and focus position according to the target tissue and selects or uses simultaneously the frequency band and focus position as required.

24. The method of claim 22, wherein the amount of deformation of said target shape is calculated by calculating an amount of deformation of the shape of the target tissue from a time variation of the data measured by the ultrasonic probe.

25. The method of claim 22, wherein in order to control the movement of said ultrasonic probe, at least one of a motor and ball screw, a linear motor, an electromagnetic drive mechanism, a mechanical spring mechanism, an air pressure, and a shape-memory alloy is used so as to move the ultrasonic probe by a predetermined distance, at predetermined speed or acceleration.

26. The method of claim 22, wherein the movement of said ultrasonic probe is controlled by generating a drive signal, giving a command and performing feedback-control with respect to the target movement position and target force generated.

27. The method of claim 22, wherein when moving the ultrasonic probe, the movement of said ultrasonic probe is controlled by taking a movement track so that the variation in acceleration becomes a minimum for the purpose of improving the estimation accuracy.

28. The method of claim 22, wherein the position of said ultrasonic probe is measured using at least one of an encoder provided for the movement mechanism, an acceleration sensor fixed to the ultrasonic probe, a spatial position sensor fixed to the ultrasonic probe, a laser rangefinder fixed to an absolute system, and a CCD camera.

29. The method of claim 22, wherein the force applied to said ultrasonic probe is measured using at least one of a distortion gauge type sensor attached to the apparatus, a load cell, a piezoelectric type force sensor, and a pressure sensor capable of measuring a pressure applied to a liquid contained in a small bag inserted between the ultrasonic probe and the target.

30. The method of claim 22, wherein viscoelasticity is estimated based on equations of motion of a physical model describing a relationship between elasticity, viscosity and inertia of the target and the force applied to the target and an amount of deformation of the target by estimating values of elasticity, viscosity and inertia in the respective regions of the target tissue from the position of the ultrasonic probe, force applied and amount of deformation of the target.

31. The method of claim 30, wherein the estimated values of elasticity, viscosity and inertia are converted to color tones and gradation, presented to a user using a method visually easy to identify or values of elasticity, viscosity and inertia about regions of the target tissue specified by the user are presented in numerical values.
